(19) United States
(12) Patent Application Publication
(10) Pub. No.: US 2007/0213670 A1
(43) Pub. Date: Sep. 13, 2007

(54) Endoscopic applicator

(76) Inventor: Jonathan B. Gabel, Randolph, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/371,835

(22) Filed: Mar. 9, 2006

(51) Int. Cl. A61M 5/178

(52) U.S. Cl. 604/164.01

(57) ABSTRACT

An endoscopic fluid applicator including an outer delivery device having a channel extending therethrough between proximal and distal ends that is defined along its length by an inner diameter, and an inner plunger device with a handle at a proximal end and an elongate plunger element extending outwardly from the handle to a distal end that is defined along its length by an outer diameter and is sized and shaped relative to the outer delivery device channel so as to be receivable therein. The elongate plunger element has a length such that, when received within the outer delivery device channel, it extends at least substantially to the distal end of the outer delivery device. Proximal end portions of the outer delivery device and inner plunger form an interference fit with one another substantially only when the elongate plunger element is fully received within the outer delivery device channel.

### BACKGROUND OF THE INVENTION

1. Field of the Invention

The present invention relates generally to surgical devices and methods, and more particularly to an endoscopic applicator particularly suitable for delivering surgical fluids, hemostatic agents or the like.

2. Background Discussion

Minimally invasive surgical procedures, such as endoscopic procedures, have become very common. These procedures typically involve one or more small incisions that provide access to the relevant internal surgical site. A trocar, cannula or the like is placed into each incision, and all surgical steps are subsequently performed through the trocar(s) using appropriately sized and designed instruments and devices.

Often times during such surgical procedures it is necessary to deliver medicinal materials, such as liquids, gels or the like, to the surgical site. Delivery to a specific location, and delivery of a specific amount of the material is often desired, such as is the case with anti-adhesion or hemostatic materials. For such materials, general washing or irrigation type delivery methods are unsuitable. Further, many such materials are costly, rendering precise application even more important.

In efforts to address these concerns, many endoscopic applicators have been developed, and those known to the inventors typically involve a long catheter-like outer tube (long enough to pass through the endoscopic trocar or port) having a channel extending therethrough between its proximal and distal ends. The surgical material, typically a fluid or gel-like material, is expressed into the channel (by any suitable means such as a syringe) for delivery to the surgical site. The proximal end includes some type of handle device to enable the user to adequately grip and manipulate the device. A second plunger-like element is sized to fit into the outer tube channel, and when slid therein expresses the material within the outer tube out through its distal end. A representative example of such a prior art applicator 100 is illustrated in FIGS. 1a-1c. The applicator 100 includes an outer delivery tube 102 having a channel 103 extending therethrough between its distal 105 and proximal 107 ends, and an inner plunger-like device 104. The inner plunger device has a substantially uniform outer diameter D₁ along most of its length, but may step up to a larger diameter D₂ at its distal end. It is sized to fit within the substantially uniform inner diameter D₃ of the channel, and a has length L₁ long enough to enable it to pass at least through to the distal end of the channel of the outer delivery tube. The relative dimensions of D₂ and D₃ allow for movement of the two pieces relative to one another, but substantially prevent the material that is being expressed from the applicator 100 to seep in between. As indicated above, during operation the desired surgical material is injected into the channel of the outer tube. The inner plunger device is subsequently inserted into the channel, and slid into the channel to thereby force the material therein to be expressed out of the distal end of the channel and into the desired location at the surgical site. Ideally, the exact amount of material injected into the channel will be dispensed to the proper location.

Some known prior art devices of the type shown in FIG. 1a-1c are packaged both pre-sterilized and pre-assembled as shown in FIG. 1a. One problem that has been encountered is that the plunger device will separate and fall out of the outer delivery tube if it is not removed from the package in precisely the right manner. One alternative would be to provide the two components in separate package compartments, which is how the disposable FloSeal Endoscopic Applicator manufactured by Baxter International, Inc. is provided. This solution, however, is undesirable in that it requires much larger packaging, which costs more to make, more to dispose of, and takes up more storage space in the hospital or the like. Another solution is to provide a non-sterilized (typically reusable) product. Although this eliminates the concern about the components accidentally falling out in the sterile field, it is disadvantageous in that the product is not immediately ready to use as shipped, but rather must be cleaned and sterilized before use.

One other known applicator is somewhat similar to that described above, but further includes toward its proximal end (towards the handle portion) an airtight membrane or seal between the outer deliver tube and the inner plunger element to prevent gases within the surgical site from escaping. This device is known as the EndoAvitine® Endscopic Delivery System, which is sold by C.R. Bard, Inc. of New Jersey, and is designed specifically to delivery a collagen hemostat, which is a solid-like material that is pre-loaded into the applicator by the manufacturer. With this device, sliding movement between the outer delivery tube and inner plunger element is possible, but only by overcoming a much larger frictional force caused by the presence of the seal. This frictional force is typically not an issue for the specified application, since, due to it's pre-loaded nature, the plunger is only used to express the solid-like material and therefore only has to travel a small distance under compressive force by the user. This type of design, however, would be unsuitable for endoscopic fluid applicators, where the plunger-like element is first entirely removed from the outer delivery tube so that the surgical fluid can be inserted therein, and subsequently re-inserted and moved substantially entirely through the outer delivery tube to express the fluid. The frictional forces generated by having such a seal at a proximal end would render the device too difficult to manipulate.

Accordingly, it would be desirable to provide a new and improved endoscopic applicator that overcomes the problems described above.

### SUMMARY OF THE INVENTION

An endoscopic fluid applicator is provided including an outer delivery device having a handle at a proximal end and an elongate tubular element extending outwardly from the handle to a distal end, where the outer delivery device has a channel extending therethrough between the proximal and distal ends, the channel being defined along its length by an inner diameter. Also included is an inner plunger device including a handle at a proximal end and an elongate plunger element extending outwardly from the handle to a distal end, where the elongate plunger element is defined along its length by an outer diameter and is sized and shaped relative to the outer delivery device channel so as to be receivable therein. The elongate plunger element further has a length such that, when received within the outer delivery device channel, it extends at least substantially to the distal end of the outer delivery device. Only proximal end portions of the outer delivery device and inner plunger device are sized and shaped so as to form an interference fit with one another substantially only when the elongate plunger element is fully received within the outer delivery device channel.

According to one embodiment, the elongate plunger element has a proximal end region, a middle region, and a distal end region, and the outer diameter is greater in the proximal end region than in the middle region. The outer delivery device channel may be substantially uniform along its length. In yet another embodiment, the outer diameter of the elongate plunger element at the middle region is approximately 3.2 mm, and the outer diameter at the proximal end region reaches approximately 4.3 mm. The outer diameter of the distal end region of the elongate plunger element may further be approximately 3.7 mm.

In yet another embodiment, the applicator further includes at least one interference element forming the interference fit as between the outer delivery device and the inner plunger device when the elongate plunger element is substantially fully received within the outer delivery device channel. In an alternate embodiment the outer diameter of the elongate tubular element may be less than approximately 5 mm.

Also provided is an endoscopic fluid applicator including an outer delivery device having a handle at a proximal end and an elongate tubular element extending outwardly from the handle to a distal end, where the outer delivery device has a channel extending therethrough between the proximal and distal ends. The applicator further includes an inner plunger device including a handle element at a proximal end and an elongate plunger element extending outwardly from the handle to a distal end, where the elongate plunger element is sized and shaped so as to be slidably receivable within the outer delivery device channel, and means at a proximal end region of the applicator for forming an interference fit between the outer delivery device and inner plunger device substantially only when elongate plunger element is fully inserted within the outer delivery device channel. The interference fit is sufficient to substantially prevent further movement of the inner plunger device relative to the outer delivery device absent exertion of a predetermined amount of force by a user.

According to alternate embodiments, the means for forming an interference fit may be an outwardly tapered proximal end region of the elongate plunger element, which may be a luer taper, may further include at least one interference element positioned between the proximal end regions of the outer delivery device channel and the elongate plunger element, or may be a recess within the handle of the inner plunger device for receiving a corresponding extension portion of the handle of the outer delivery device.

According to various other embodiments, the outer diameter of the elongate tubular element may be less than approximately 5 mm and/or the inner diameter of the outer delivery device channel may be approximately 3.8 mm.

An endoscopic applicator is also provided having an outer delivery device including a handle at a proximal end and an elongate tubular element extending outwardly from the handle to a distal end, where the outer delivery device has a channel extending therethrough between the proximal and distal ends, and an inner plunger device including a handle element at a proximal end and an elongate plunger element extending outwardly from the handle to a distal end, where the elongate plunger element is sized and shaped so as to be slidably receivable within the outer delivery device channel. Also included is a means at a proximal end region of the applicator for forming an threaded fit between the outer delivery device and inner plunger device when the elongate plunger element is substantially fully inserted within the outer delivery device channel. The threaded fit is sufficient to substantially prevent further movement of the inner plunger device relative to the outer delivery device absent further manipulation by a user.

The means for forming an threaded fit further comprises male and female thread forms at the proximal end region of the inner plunger device and outer delivery device channel.

According to one embodiment, the male and female thread forms are Luer taper threads. In another embodiment, the outer diameter of the elongate tubular element is less than 5 mm, and in yet another embodiment, the inner diameter of the outer delivery device channel is approximately 3.8 mm.

### BRIEF DESCRIPTION OF THE DRAWINGS

FIGS. 1a-1c are side-views illustrating an exemplary prior art design and various components thereof;

FIGS. 2a and 2b are side-views illustrating components of one embodiment of an applicator according to the present invention;

FIG. 3 is a cross sectional view of the components of FIGS. 2a and 2b when assembled together;

FIG. 4 is a side view of the components of FIGS. 2a and 2b when assembled together; and

FIGS. 5a-5e illustrate alternate means by which to form an interference fit as between the outer delivery device and inner plunger device.

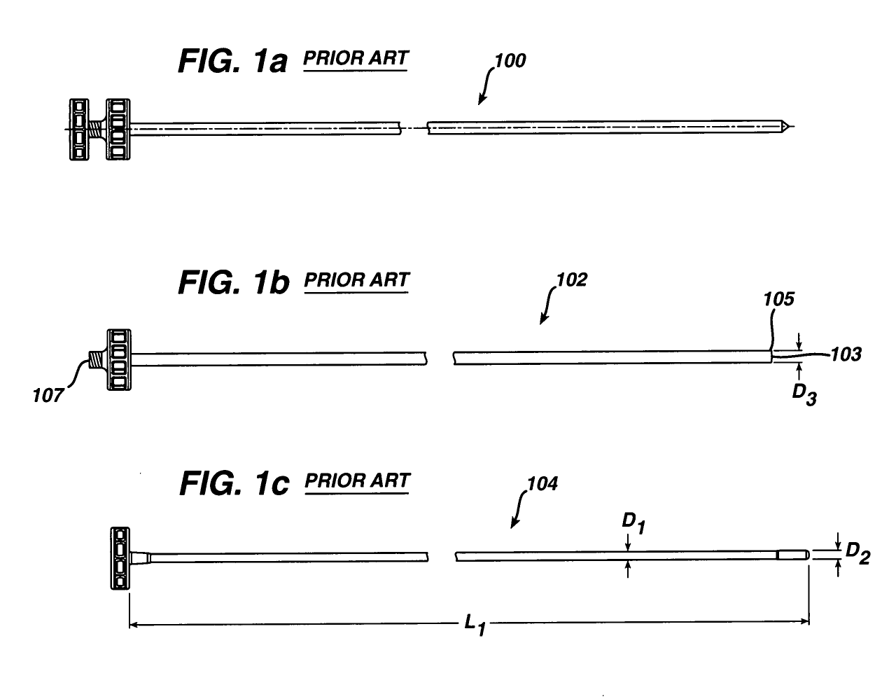

### DETAILED DESCRIPTION OF THE INVENTION

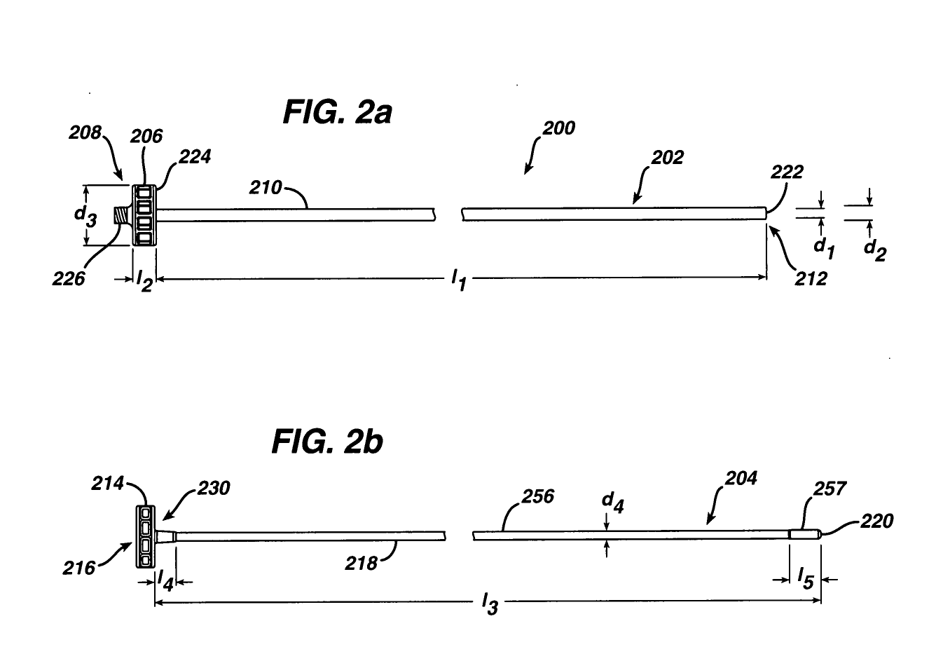

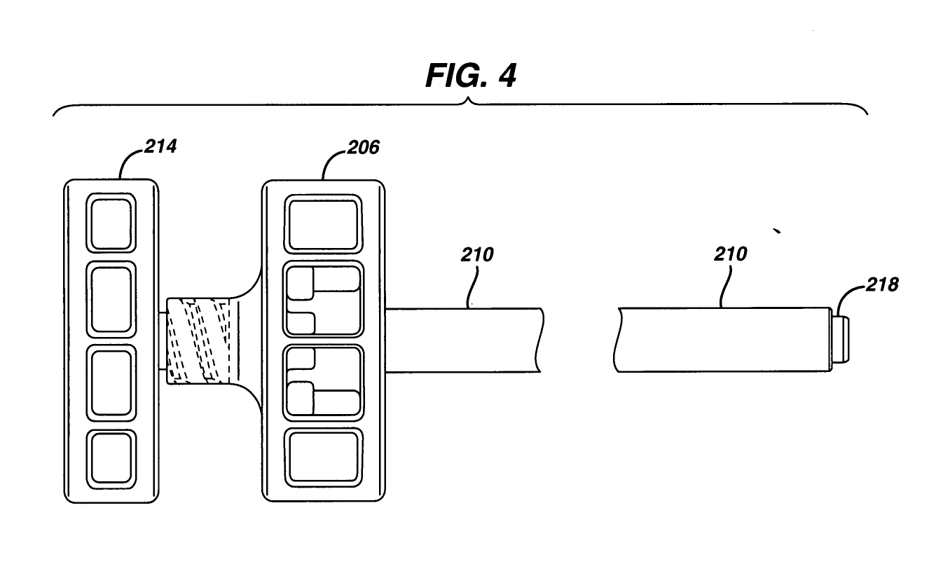

Referring now to FIGS. 2-4, which illustrate a preferred embodiment of the present invention, the applicator 200 includes an outer delivery device 202 and an inner plunger device 204. The outer delivery device 202 has a handle element 206 at a proximal end 208 thereof, and an elongate tubular element 210 extending outwardly from the handle element to a distal end 212. Similarly, the inner plunger device 204 has a handle element 214 at a proximal end 216 thereof, and an elongate plunger element 218 extending outwardly from the handle element to a distal end 220.

The outer delivery device includes a channel 222 extending therethrough between the proximal 208 and distal 212 ends, with the size of the channel being defined by an inner diameter d₁ of the outer delivery device. Further, the device and channel therethrough include a proximal end region 250, a middle region 251 and a distal end region 252. Although the term “diameter” is used, it is to be understood that the term is not meant to be limited to circular cross-sections, although circular is the preferred embodiment. Further, the term “diameter” is not intended to require a constant diameter along the length of the device, as will be described further below.

The outer diameter d₂ and length l₁ of the elongate tubular member 210 must be suitable for insertion through an endoscopic port such as a cannula or trocar. According to a preferred embodiment, the inner diameter d₁ is approximately 3.8 mm, the outer diameter d₂ is approximately 4.8 mm, and the length l₁ is approximately 34 cm. With regard to handle element 206, any suitable configuration may be used that adequately allows the user to grasp and manipulate the outer delivery device. In the illustrated embodiment, the handle has a substantially disc-like portion 224 having a length l₂ of approximately 10 mm, and a diameter d₃ of approximately 2.5 cm, and an extension portion 226 at its proximal end.

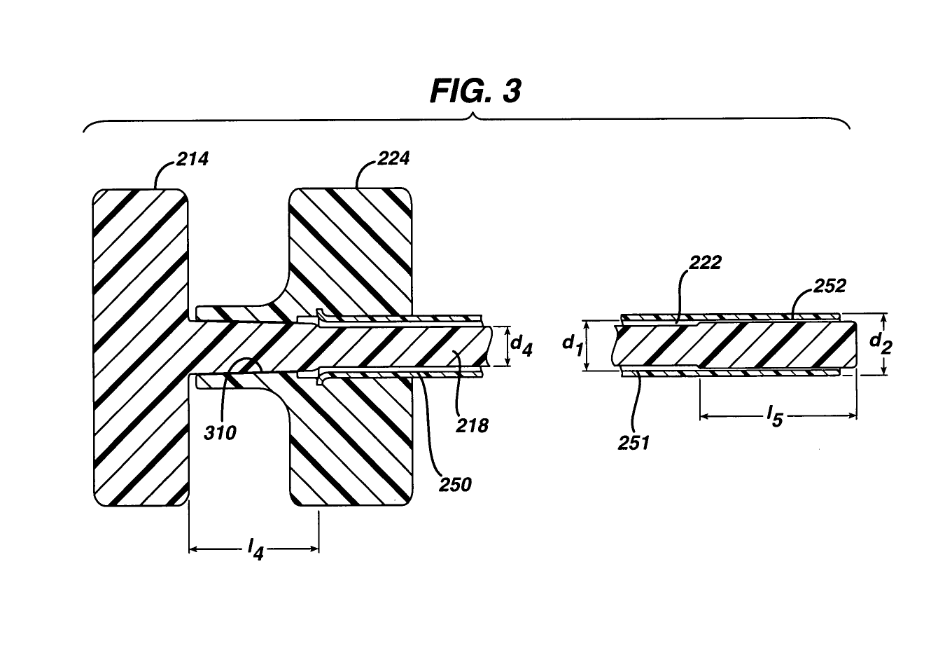

The elongate plunger element 218 of the inner plunger device 204 is dimensioned relative to the outer delivery device channel so as to be receivable therein, and slidable therein along a substantial portion of its length. In the illustrated embodiment, the length l₃ is further sized such that, when the elongate plunger element is inserted into the outer delivery channel, the distal end 220 of the elongate plunger element is at least substantially aligned with the distal end 212 of the outer delivery device, and preferably extends slightly beyond, as illustrated in greater detail in FIG. 3. The elongate plunger element is also defined by an outer diameter along its length l₃ and includes a proximal end region 230, a middle region 256 and a distal end region 257. In the illustrated embodiment, the outer diameter d₄ along most of the length is approximately 3.2 mm, whereas the outer diameter expands to approximately 3.7 mm along a distal portion of the length l₅. This allows the distal end of the elongate plunger element to have a closer tolerance relative to the channel of the outer delivery device, to enable fluid within the channel to more precisely be expressed with little or no leakage between the two elements and without significantly affecting the force required to slide the inner plunger element within the channel.

Referring now to the proximal end portion 230 of the elongate plunger element, in the illustrated embodiment shown in greater detail in FIG. 3, the diameter d₄ also expands outwardly in a tapered fashion, such as a luer taper, towards the proximal end. In a preferred embodiment, the expansion takes place along a length l₄ of approximately 10 mm, and to a maximum diameter of approximately 4.3 mm. This tapered proximal end portion 230 forms an interference fit with the outer delivery device, thereby preventing further movement relative to one another absent exertion of force by a user to separate the two. In this manner, it is ensured that the devices will not accidentally separate from one another when removed from the packaging as described above.

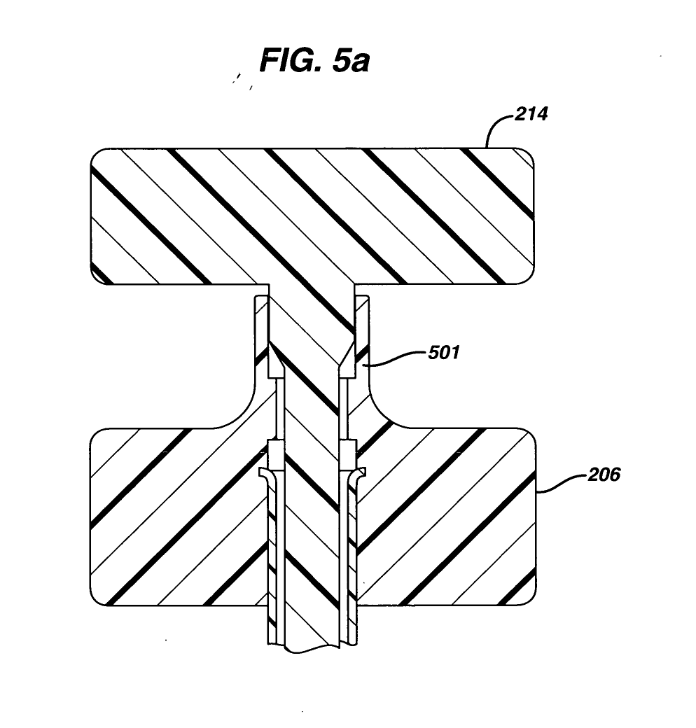

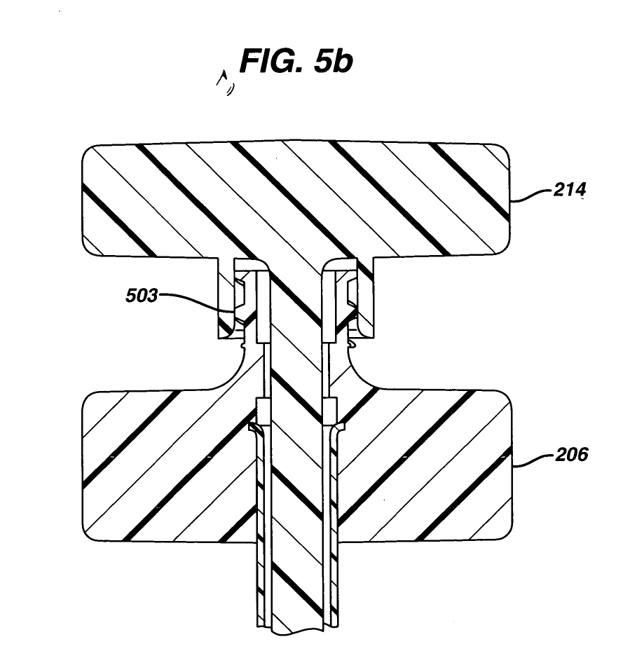

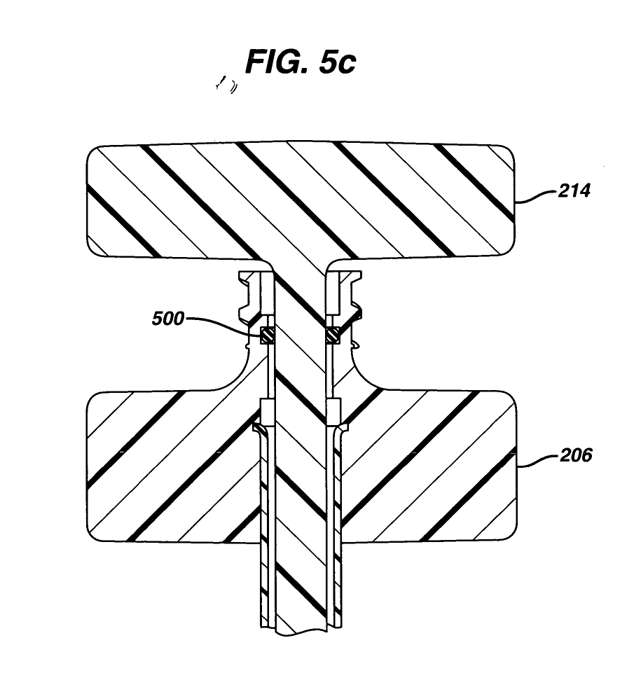

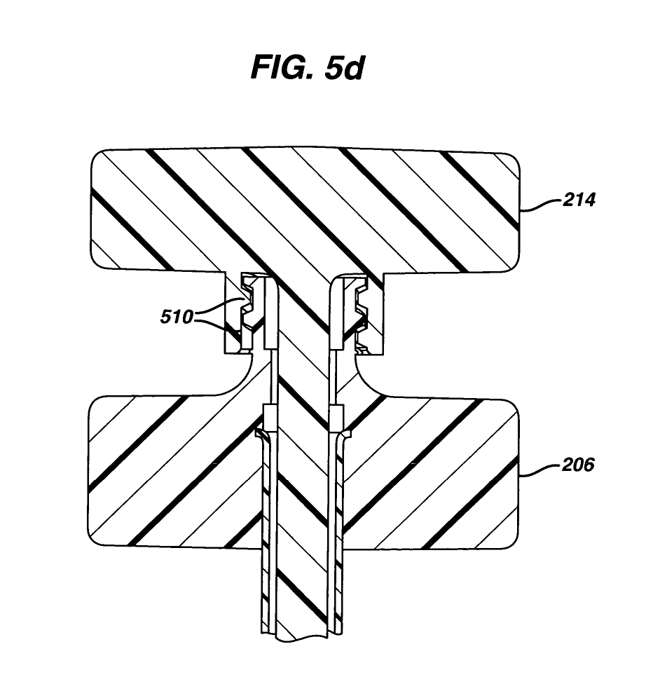

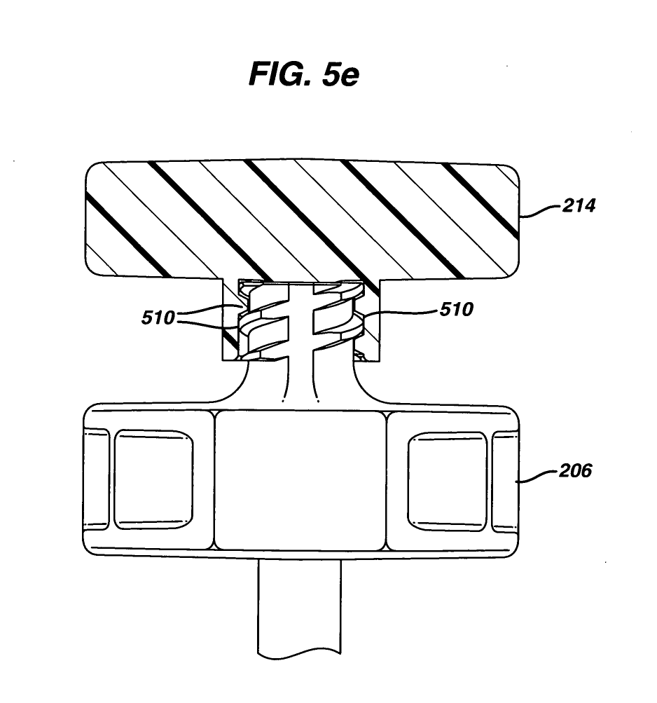

Although the illustrated embodiment shows an outwardly tapered or flared proximal end portion of the elongate plunger element, it is to be understood that any suitable means for forming an interference or locking fit as between the proximal end portions of the outer delivery device and inner plunger device can be used. For example, the respective handle portions could be appropriately configured so as to form an interference fit therebetween as shown in FIGS. 5a (at reference numeral 501) and 5b (at reference numeral 503). Other means could include additional interference elements 500, such as gaskets or the like, that assist in forming an interference fit between the handle portions (i.e., FIG. 5c, or as between the outer delivery device and inner plunger device. Other means could also include complementary engaging threads 510 as shown in FIGS. 5d and 5e.

It will be apparent from the foregoing that, while particular forms of the invention have been illustrated and described, various modifications can be made without departing from the spirit and scope of the invention. Accordingly, it is not intended that the invention be limited, except as by the appended claims.

What is claimed is:

1. An endoscopic fluid applicator comprising:
an outer delivery device having a handle at a proximal end and an elongate tubular element extending outwardly from the handle to a distal end, wherein the outer delivery device has a channel extending therethrough between the proximal and distal ends, the channel being defined along its length by an inner diameter;
an inner plunger device including a handle at a proximal end and an elongate plunger element extending outwardly from the handle to a distal end, wherein the elongate plunger element is defined along its length by an outer diameter and is sized and shaped relative to the outer delivery device channel so as to be receivable therein, and wherein the elongate plunger element has a length such that, when received within the outer delivery device channel, it extends at least substantially to the distal end of the outer delivery device;
wherein only proximal end portions of the outer delivery device and inner plunger device are sized and shaped so as to form an interference fit with one another substantially only when the elongate plunger element is fully received within the outer delivery device channel.

2. The applicator according to claim 1, wherein the elongate plunger element has a proximal end region, a middle region, and a distal end region, and wherein the outer diameter is greater in the proximal end region than in the middle region.

3. The applicator according to claim 2, wherein the inner diameter of the outer delivery device channel is substantially uniform along its length.

4. The applicator according to claim 2, wherein the outer diameter of the elongate plunger element at the middle region is approximately 3.2 mm, and the outer diameter at the proximal end region is reaches approximately 4.3 mm.

5. The applicator according to claim 4, wherein the outer diameter of the distal end region of the elongate plunger element is approximately 3.7 mm.

6. The applicator according to claim 1, further comprising at least one interference element forming said interference fit as between the outer delivery device and the inner plunger device when the elongate plunger element is substantially fully received within the outer delivery device channel.

7. The applicator according to claim 1, wherein an outer diameter of the elongate tubular element is less than approximately 5 mm.

8. An endoscopic fluid applicator comprising:
an outer delivery device including a handle at a proximal end and an elongate tubular element extending outwardly from the handle to a distal end, wherein the outer delivery device has a channel extending therethrough between the proximal and distal ends;
an inner plunger device including a handle element at a proximal end and an elongate plunger element extending outwardly from the handle to a distal end, wherein the elongate plunger element is sized and shaped so as to be slidably receivable within the outer delivery device channel; and
means at a proximal end region of the applicator for forming an interference fit between the outer delivery device and inner plunger device substantially only when elongate plunger element is fully inserted within the outer delivery device channel, the interference fit being sufficient to substantially prevent further movement of the inner plunger device relative to the outer delivery device absent exertion of a predetermined amount of force by a user.

9. The applicator according to claim 8, wherein the means for forming an interference fit is an outwardly tapered proximal end region of the elongate plunger element.

10. The applicator according to claim 9, wherein the tapered end region is a luer taper.

11. The applicator according to claim 8, wherein the means for forming an interference fit further comprises at least one interference element positioned between the proximal end regions of the outer delivery device channel and the elongate plunger element.

12. The applicator according to claim 8, wherein the means for forming an interference fit comprises a recess within the handle of the inner plunger device for receiving a corresponding extension portion of the handle of the outer delivery device.

13. The applicator according to claim 8, wherein the outer diameter of the elongate tubular element is less than approximately 5 mm.

14. The applicator according to claim 12, wherein the inner diameter of the outer delivery device channel is approximately 3.8 mm.

15. An endoscopic applicator comprising:
an outer delivery device including a handle at a proximal end and an elongate tubular element extending outwardly from the handle to a distal end, wherein the outer delivery device has a channel extending therethrough between the proximal and distal ends;
an inner plunger device including a handle element at a proximal end and an elongate plunger element extending outwardly from the handle to a distal end, wherein the elongate plunger element is sized and shaped so as to be slidably receivable within the outer delivery device channel; and
means at a proximal end region of the applicator for forming an threaded fit between the outer delivery device and inner plunger device when the elongate plunger element is substantially fully inserted within the outer delivery device channel, the threaded fit being sufficient to substantially prevent further movement of the inner plunger device relative to the outer delivery device absent further manipulation by a user.

16. The applicator according to claim 15, where the means for forming an threaded fit further comprises male and female thread forms at the proximal end region of the inner plunger device and outer delivery device channel.

17. The applicator according to claim 16, wherein the male and female thread forms are Luer taper threads.

18. The applicator according to claim 15, wherein the outer diameter of the elongate tubular element is less than 5 mm.

19. The applicator according to claim 15, wherein the inner diameter of the outer delivery device channel is approximately 3.8 mm.
